(19) United States
(12) Patent Application Publication
(10) Pub. No.: US 2021/0004445 A1
(43) Pub. Date: Jan. 7, 2021

(54) FINGERPRINT MODULE, FINGERPRINT IDENTIFICATION SYSTEM, CONTROL METHOD, AND SMART LOCK

(71) Applicant: Shenzhen Goodix Technology Co., Ltd., Shenzhen (CN)

(72) Inventors: Qingbin LI, Shenzhen (CN); Luming ZHOU, Shenzhen (CN)

(73) Assignee: Shenzhen Goodix Technology Co., Ltd., Shenzhen (CN)

(21) Appl. No.: 17/029,428

(22) Filed: Sep. 23, 2020

Related U.S. Application Data
(63) Continuation of application No. PCT/CN2019/074496, filed on Feb. 1, 2019.

(51) Int. Cl. G06F 21/32; G06K 9/00; G06F 1/3231

(52) U.S. Cl. CPC G06F 21/32; G06K 9/00087; G06K 2009/00939; G06K 9/00906; G06F 1/3231; G06K 9/0012

(57) ABSTRACT

A fingerprint module, a fingerprint identification system, a control method and a smart lock. The fingerprint module includes: a fingerprint sensor, an MOC, and a key member, and the key member is connected to the fingerprint sensor. The key member is configured to generate an interrupt signal according to a touch operation of a user on the key member to wake up the main control unit, thereby reducing power consumption of the main control unit.

### CROSS-REFERENCE TO RELATED APPLICATIONS

This application is a continuation of International Application No. PCT/CN2019/074496, filed on Feb. 1, 2019, the disclosure of which is hereby incorporated by reference in its entirety.

### TECHNICAL FIELD

The present disclosure relates to the field of fingerprint identification technologies, and in particular, to a fingerprint module, a fingerprint identification system, a control method and a smart lock.

### BACKGROUND

Smart locks are ubiquitous in people's lives, which can be unlocked in various ways, such as an unlocking method based on Near Field Communication (NFC), a digital password unlocking method, a fingerprint password unlocking method, etc. A smart lock can involve one or more unlocking methods.

At present, a smart lock with fingerprint identification function includes: a main control unit, a fingerprint sensor and a flash (FLASH). A fingerprint identification process of the smart lock is: the fingerprint sensor collects a fingerprint image and sends the fingerprint image to the main control unit, the main control unit matches the acquired fingerprint image with a fingerprint template in the flash to obtain a matching result, and when matching is successful, the main control unit in the smart lock controls an actuator to drive the smart lock to be in an open state.

In the prior art, the fingerprint identification process is mainly implemented by the main control unit, which will cause a problem of excessive power consumption of the main control unit.

### SUMMARY

The present disclosure provides a fingerprint module, a fingerprint identification system, a control method and a smart lock, so that power consumption of the smart lock can be reduced.

In a first aspect, the present disclosure provides a fingerprint module connected to a main control unit, the fingerprint module includes: a fingerprint sensor, a match on chip (MOC), and a key member, and the key member is connected to the fingerprint sensor. The fingerprint sensor is configured to collect fingerprint information of a verification target, and output the fingerprint information to the MOC. The MOC is configured to authenticate the fingerprint information, and output an authentication result to the main control unit. The key member is configured to generate an interrupt signal according to a touch operation of a user on the key member, to wake up the main control unit.

In a possible design, the MOC and/or the fingerprint sensor is further configured to receive a first control instruction sent by the main control unit, and enter a power-up state according to the first control instruction.

In a possible design, the MOC is further configured to control the key member to be in a power-down state.

In a possible design, the MOC is connected to the key member through a control switch. The MOC is specifically configured to control the key member to be in the power-down state through the control switch when the MOC is in the power-up state.

In a possible design, the MOC and/or the fingerprint sensor is further configured to receive a second control instruction sent by the main control unit, and enter a power-down state according to the second control instruction.

In a possible design, the MOC is further configured to control the key member to be in a power-up state.

In a possible design, the MOC is connected to the key member through a control switch. The MOC is configured to trigger the control switch to output a low-level signal, when the MOC is in the power-down state, where the low-level signal is used to trigger the key member to be in the power-up state.

In a possible design, the fingerprint module further includes: a flash. The flash includes at least one fingerprint template. The MOC is specifically configured to match the fingerprint information with the at least one fingerprint template, and output an authentication result.

In a possible design, the authentication result is a result of successful authentication or a result of failed authentication.

In a possible design, the fingerprint module further includes a living body detection module. The living body detection module is configured to detect at least one living body index of the verification target, and output the at least one living body index to the MOC. The MOC is further configured to determine whether the verification target is a living body according to the at least one living body index.

In a possible design, the MOC is configured to output an authentication result of successful authentication when the fingerprint information is successfully authenticated and the verification target is the living body.

In a possible design, the living body index includes at least one of: blood oxygen or heart rate.

In a second aspect, the present disclosure provides a fingerprint identification system, including: a main control unit and the fingerprint module as described in the first aspect or optional designs of the first aspect.

In a possible design, the main control unit is specifically configured to: send a second control instruction to the MOC and/or the fingerprint sensor when the main control unit does not receive information within a preset period of time, and the second control instruction is configured to control the MOC and/or the fingerprint sensor to enter a power-down state.

In a third aspect, the present disclosure provides a control method based on a fingerprint identification system, the fingerprint identification system includes: a main control unit and a fingerprint module, and the fingerprint module includes: a fingerprint sensor, a match on chip (MOC) and a key member, the key member is connected to the fingerprint sensor. Correspondingly, the method includes: the key member generates an interrupt signal according to a touch operation of a user on the key member, to wake up the main control unit, the main control unit controls the fingerprint sensor and/or the MOC to power up, the fingerprint sensor collects fingerprint information of a verification target, and outputs the fingerprint information to the MOC, and the MOC authenticates the fingerprint information and outputs an authentication result to the main control unit.

In a fourth aspect, the present disclosure provides a smart lock, including: the fingerprint identification system as described in the third aspect or optional designs of the third aspect.

The present disclosure provides a fingerprint module, a fingerprint identification system, a control method and a smart lock, the fingerprint module includes: a fingerprint sensor, an MOC and a key member, and the key member is connected to the fingerprint sensor. The key member is configured to generate an interrupt signal according to a touch operation of a user on the key member, to wake up the main control unit. That is, the MOC can implement a fingerprint identification process, and thus power consumption of the main control unit can be reduced. When the MOC and/or the fingerprint sensor is in a power-off state, the main control unit sends a first control instruction to the MOC and/or the fingerprint sensor, so that the MOC and/or the fingerprint sensor enters a power-up state according to the first control instruction. Based on this, a smart lock including the fingerprint module can perform a fingerprint identification process, etc. Optionally, the MOC is configured to control the key member to be in a power-down state, when the MOC is in the power-up state, which can further reduce power consumption of the smart lock. When the main control unit does not receive information within a preset period of time, the main control unit sends a second control instruction to the MOC and/or fingerprint sensor, so that the MOC and/or the fingerprint sensor enters the power-down state according to the second control instruction. When the fingerprint module is applied to the smart lock, power consumption of the smart lock can thus be reduced. Optionally, when the MOC enters the power-down state, the MOC is further configured to control the key member to be in the power-up state, thereby ensuring that the smart lock can obtain the operation of a user at any time.

### BRIEF DESCRIPTION OF DRAWINGS

In order to describe the technical solutions in embodiments of the present disclosure or in the prior art more clearly, the following briefly introduces the accompanying drawings needed for describing the embodiments or the prior art. Apparently, the accompanying drawings in the following description are merely some embodiments of the present disclosure, and persons of ordinary skill in the art may still derive other accompanying drawings from these accompanying drawings without creative effort.

FIG. 1 is a schematic diagram of a fingerprint module according to an embodiment of the present disclosure;

FIG. 2 is a schematic diagram of a fingerprint module according to another embodiment of the present disclosure;

FIG. 3 is a schematic diagram of a fingerprint module according to a still another embodiment of the present disclosure;

FIG. 4 is a schematic diagram of a fingerprint identification system according to an embodiment of the present disclosure;

FIG. 5 is a schematic diagram of a fingerprint identification system according to another embodiment of the present disclosure;

FIG. 6 is a schematic diagram of a fingerprint identification system according to still another embodiment of the present disclosure;

FIG. 7 is a flowchart of a control method based on a fingerprint identification system according to an embodiment of the present disclosure; and

FIG. 8 is a flowchart of a control method based on a fingerprint identification system according to another embodiment of the present disclosure.

### DETAILED DESCRIPTION OF EMBODIMENTS

In order to make the purposes, technical solutions and advantages in embodiments of the present disclosure more clearly, the technical solutions in the embodiments of the present disclosure will be described clearly and completely below in combination with accompanying drawings in the embodiments of the present disclosure. Apparently, the described accompanying drawings in the following description are merely some embodiments of the present disclosure, rather than all of them. All other embodiments obtained by those skilled in the art without paying creative effort shall fall within the scope of protection of the present disclosure.

The terms “first”, “second”, “third”, “fourth” and/or the like (if any) in the description, claims and the drawings of the present disclosure are used to distinguish similar objects, and are not necessarily used to describe specific order or sequence. It should be understood that the numerals used as such are interchangeable under appropriate circumstances, so that the embodiments described herein, for example, can be implemented in alternative order other than those illustrated or described herein. In addition, the terms “include” and “have” and any variations thereof are intended to cover non-exclusive inclusion. For example, a process, a method, a system, a product or an apparatus that includes a series of steps or units do not have to be limited to those steps or units expressly listed, but may instead include other steps or units not expressly listed, or steps or units inherent to the process, method, product or apparatus.

As mentioned above, in the prior art, the fingerprint identification process is implemented by a main control unit, which will cause a problem of excessive power consumption of the main control unit. In order to solve this technical problem, the present disclosure provides a fingerprint module, a fingerprint identification system, a control method and a smart lock.

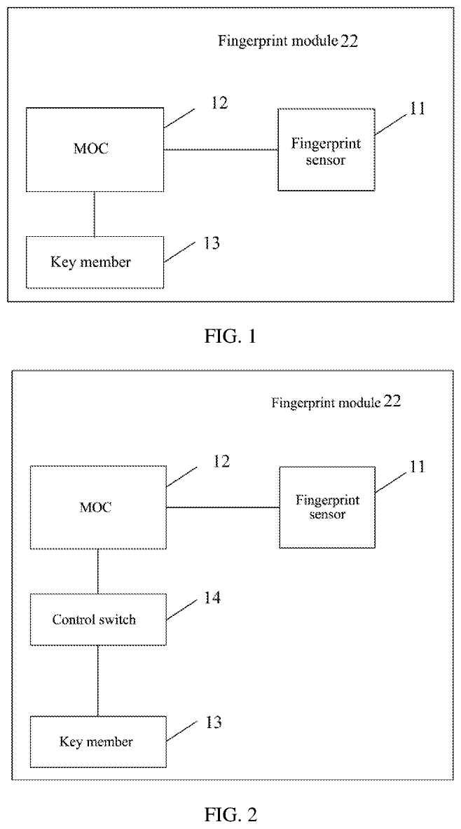

FIG. 1 is a schematic diagram of a fingerprint module according to an embodiment of the present disclosure, and the fingerprint module is connected to a main control unit. As shown in FIG. 1, a fingerprint module 22 includes a fingerprint sensor 11, a match on chip (Match On Chip, MOC) 12 and a key member 13, and the key member 13 is connected to the fingerprint sensor 11. The fingerprint sensor 11 is configured to collect fingerprint information of a verification target and output the fingerprint information to the MOC 12. The MOC 12 is configured to authenticate the fingerprint information and output an authentication result to the main control unit.

The key member 13 is configured to generate an interrupt signal according to a touch operation of a user on the key member 13, to wake up the main control unit. When the main control unit does not receive information, for example, the authentication result sent by the MOC 12, within a preset period of time, the main control unit enters a sleep state. When the above fingerprint module is applied to a smart lock, power consumption of the smart lock can be reduced in this way. When the key member 13 acquires a touch operation of a user, the key member 13 generates an interrupt signal, which is used to wake up the main control unit, so that the main control unit enters a working state.

In a possible design, the main control unit may have a GPIO interface, and the key member 13 may transmit the interrupt signal to the main control unit through the GPIO interface.

When the main control unit is woken up, in this case, if the MOC 12 and/or fingerprint sensor 11 is in a power-off state, the main control unit sends a first control instruction to the MOC and/or fingerprint sensor to cause the MOC 12 and/or the fingerprint sensor 11 to enter a power-up state according to the first control instruction.

In a possible design, the MOC 12 is configured to control the key member 13 to be in the power-down state, when the MOC 12 is in the power-up state. Specifically, when the MOC 12 is in the power-up state, the MOC 12 can perform a fingerprint identification process. In this case, the key member 13 does not need to work. In order to save the power consumption of the smart lock, the MOC 12 can control the key member 13 to be in a power-down state.

In a possible design, the main control unit may be a micro controller unit (Micro Controller Unit, MCU).

In a possible design, the main control unit has a universal asynchronous receiver transmitter (Universal Asynchronous Receiver Transmitter, UART) interface, and communicates with the MOC 12 through the UART interface.

In a possible design, there is a low dropout regulator (Low Dropout Regulator, LDO) switch connected between the main control unit and the fingerprint module, and the main control unit has a general purpose input output (General Purpose Input Output, GPIO) interface, and controls a state of the LDO switch through the GPIO interface.

In a possible design, the MOC 12 and the fingerprint sensor 11 are connected through a serial peripheral interface (Serial Peripheral Interface, SPI) interface, and the MOC 12 and the fingerprint sensor 11 can communicate through the SPI interface.

In a possible design, the key member 13 may be a ring key, which surrounds the fingerprint sensor 11. In a possible design, FIG. 2 is a schematic diagram of a fingerprint module according to another embodiment of the present disclosure, where the MOC 12 is connected to the key member 13 through a control switch 14.

In a possible design, the MOC 12 is connected to the key member 13 through the control switch 14, and the MOC 12 is specifically configured to control the key member 13 to be in a power-down state through the control switch 14, when the MOC 12 is in a power-up state.

In a possible design, the MOC 12 has a GPIO interface, and the MOC 12 can control a state of the control switch 14 between the MOC 12 and the key member 13 through the GPIO interface.

The embodiment of the present disclosure provides a fingerprint module, including: a fingerprint sensor, an MOC and a key member. The key member is connected to the fingerprint sensor. The fingerprint sensor is configured to collect fingerprint information of a verification target, and output the fingerprint information to the MOC. The MOC is configured to authenticate the fingerprint information, and output an authentication result to the main control unit. The key member is configured to generate an interrupt signal according to a touch operation of a user on the key member, to wake up the main control unit. That is, the MOC can implement a fingerprint identification process, so that power consumption of the main control unit can be reduced. Optionally, when the MOC and/or the fingerprint sensor is in a power-off state, the main control unit sends a first control instruction to the MOC and/or the fingerprint sensor, so that the MOC and/or the fingerprint sensor enters a power-up state according to the first control instruction. Based on this, a smart lock including the fingerprint module can perform the fingerprint identification process, etc. Optionally, the MOC is configured to control the key member to be in a power-down state, when the MOC 12 is in the power-up state. Therefore, when the fingerprint module is applied to the smart lock, power consumption of the smart lock can be reduced.

On the basis of the previous embodiment, further, when the main control unit does not receive information, for example, the authentication result sent by the MOC 12, within a preset period of time, the main control unit sends a second control instruction to the MOC 12 and/or the fingerprint sensor 11, so that the MOC 12 and/or the fingerprint sensor 11 enters the power-down state according to the second control instruction. The above preset period of time may be set according to an actual condition, for example, the preset period of time is set to 10 minutes, 15 minutes, etc., which is not limited in the present disclosure.

In a possible design, when the MOC 12 enters the power-down state, the MOC 12 is further configured to control the key member 13 to be in the power-up state.

In a possible design, the MOC 12 is connected to the key member 13 through the control switch 14; when the MOC 12 is in the power-down state, the MOC 12 is configured to trigger the control switch 14 to output a low-level signal, where the low-level signal is used to trigger the key member to be in the power-up state.

Or, in another possible design, when the MOC 12 is in the power-down state, the MOC 12 is configured to trigger the control switch 14 to output a high-level signal, where the high-level signal is used to trigger the key member to be in the power-up state.

In the above solution, the MOC 12 is configured to control the key member 13 to be in the power-up state. Actually, the main control unit may also directly control the key member 13 to be in the power-up state.

In a possible design, the main control unit is connected to the key member through the control switch. When the main control unit is in the sleep state, the main control unit triggers the control switch to output a low-level signal, where the low-level signal is used to trigger the key member to be in the power-up state.

Or, in another possible design, when the main control unit is in the sleep state, the main control unit triggers the control switch to output a high-level signal; where the high-level signal is used to trigger the key member to be in the power-up state.

The embodiment of the present disclosure provides a fingerprint module, including: a fingerprint sensor, an MOC and a key member, and the key member is connected to the fingerprint sensor. When the main control unit does not receive information within a preset period of time, the main control unit sends a second control instruction to the MOC and/or fingerprint sensor, so that the MOC and/or the fingerprint sensor enters the power-down state according to the second control instruction. When the fingerprint module is applied to the smart lock, power consumption of the smart lock can be reduced. Optionally, when the MOC enters the power-down state, the MOC is further configured to control the key member to be in the power-up state, thereby ensuring that the smart lock can obtain an operation of a user at any time.

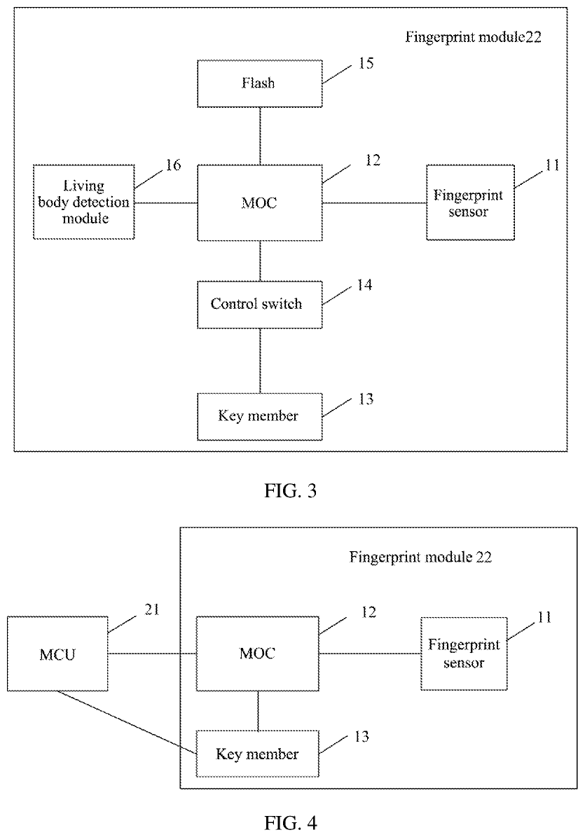

On the basis of any of the previous embodiments, further, the fingerprint module further includes: a flash (FLASH) and/or a living body detection module. The following is a detailed description of the fingerprint module including a flash and a living body detection module. FIG. 3 is a schematic diagram of a fingerprint module according to still another embodiment of the present disclosure. As shown in FIG. 3, the fingerprint module includes: a flash 15 and a living body detection module 16, where the flash 15 includes at least one fingerprint template; and correspondingly, the MOC 12 is specifically configured to match the fingerprint information with the at least one fingerprint template, and output an authentication result to the main control unit. The authentication result is a result of successful authentication or a result of failed authentication.

In a possible design, the living body detection module is configured to detect at least one living body index of the verification target, and output the at least one living body index to the MOC. The MOC is further configured to determine whether the verification target is a living body according to the at least one living body index. The living body index includes at least one of: blood oxygen or heart rate. For example: when determining that a heart rate of the verification target is within a preset range, the MOC determines the verification target is a living body.

When the fingerprint information is successfully authenticated and the verification target is the living body, the MOC may output an authentication result of successful authentication to the main control unit. Conversely, when the fingerprint information fails to be authenticated or the verification target is not the living body, the MOC may output an authentication result of failed authentication to the main control unit.

In a possible design, after the main control unit is woken up, if at least one of the MOC 12, the fingerprint sensor 11, the flash 15 and the living body detection module 16 is in the power-off state, the main control unit will send a first control instruction to at least one of the MOC 12, the fingerprint sensor 11, the flash 15 and the living body detection module 16, so that the at least one of the MOC 12, the fingerprint sensor 11, the flash 15 and the living body detection module 16 will enter the power-up state according to the first control instruction.

When the main control unit does not receive information, for example, the authentication result sent by the MOC 12, within a preset period of time, and at least one of the MOC 12, the fingerprint sensor 11, the flash 15 and the living body detection module 16 is in the power-up state, the main control unit may send a second control instruction at least one of the MOC 12, the fingerprint sensor 11, the flash 15 and the living body detection module 16, so that at least one of the MOC 12, the fingerprint sensor 11, the flash 15 and the living body detection module 16 enters the power-down state according to the second control instruction.

The embodiment of the present disclosure provides a fingerprint module, which includes a fingerprint sensor, an MOC, a key member, a FLASH and a living body detection module. After the main control unit is woken up, the main control unit may send a first control instruction to at least one of the MOC, the fingerprint sensor, the flash and the living body detection module, so that at least one of the MOC, the fingerprint sensor, the flash and the living body detection module will enter the power-up state according to the first control instruction. Based on this, a smart lock including the fingerprint module can perform a fingerprint identification process, etc. Optionally, the MOC is configured to control the key member to be in a power-down state, when the MOC is in the power-up state, which can further reduce power consumption of the smart lock. When the main control unit does not receive information within a preset period of time, the main control unit will send a second control instruction to at least one of the MOC, the fingerprint sensor, the flash and the living body detection module, so that at least one of the MOC, the fingerprint sensor, the flash and the living body detection module enters the power-down state according to the second control instruction. When the fingerprint module is applied to the smart lock, power consumption of the smart lock can be reduced. Optionally, when the MOC enters the power-down state, the MOC is further configured to control the key member to be in the power-up state, thereby ensuring that the smart lock can obtain an operation of a user at any time.

FIG. 4 is a schematic diagram of a fingerprint identification system according to an embodiment of the present disclosure, where the fingerprint identification system may be applied to but not limited to a smart lock. As shown in FIG. 4, the fingerprint identification system includes: a main control unit 21 and a fingerprint module 22. The fingerprint module 22 includes a fingerprint sensor 11, a MOC 12 and a key member 13, and the key member 13 is connected to the fingerprint sensor 11. The fingerprint sensor 11 is configured to collect fingerprint information of a verification target and output the fingerprint information to the MOC 12. The MOC 12 is configured to authenticate the fingerprint information and output an authentication result to the main control unit 21.

The key member 13 is configured to generate an interrupt signal according to a touch operation of a user on the key member 13, to wake up the main control unit 21. When the main control unit 21 does not receive information, for example, the authentication result sent by the MOC 12, within a preset period of time, the main control unit 21 enters a sleep state. When the above fingerprint module is applied to a smart lock, power consumption of the smart lock can be reduced in this way. However, when the key member 13 acquires the touch operation of a user, the key member 13 generates an interrupt signal, and the interrupt signal is used to wake up the main control unit 21 so that the main control unit 21 enters a working state.

In a possible design, the main control unit 21 may have a GPIO interface, and the key member 13 may transmit the interrupt signal to the main control unit 21 through the GPIO interface.

When the main control unit 21 is woken up, in this case, if the MOC 12 and/or fingerprint sensor 11 is in a power-off state, the main control unit 21 sends a first control instruction to the MOC and/or fingerprint sensor, so that the MOC 12 and/or the fingerprint sensor 11 enters a power-up state according to the first control instruction.

In a possible design, the MOC 12 is configured to control the key member 13 to be in the power-down state, when the MOC 12 is in the power-up state. Specifically, when the MOC 12 is in the power-up state, the MOC 12 can perform a fingerprint identification process. In this case, the key member 13 does not need to work. In order to save the power consumption of the smart lock, the MOC 12 can control the key member 13 to be in a power-down state.

In a possible design, the main control unit 21 may be a MCU.

In a possible design, the main control unit 21 has a UART interface, and communicates with the MOC 12 through the UART interface.

In a possible design, there is a LDO switch connected between the main control unit 21 and the fingerprint module, and the main control unit 21 has a GPIO interface, and controls a state of the LDO switch through the GPIO interface.

In a possible design, the MOC 12 and the fingerprint sensor 11 are connected through an SPI interface, and the MOC 12 and the fingerprint sensor 11 can communicate through the SPI interface.

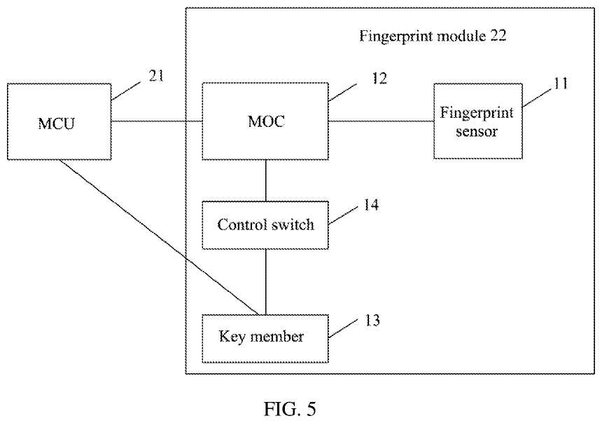

In a possible design, the key member 13 may be a ring key, which surrounds the fingerprint sensor 11. In a possible design, FIG. 5 is a schematic diagram of a fingerprint identification system according to another embodiment of the present disclosure, where the MOC 12 is connected to the key member 13 through a control switch 14 as shown in FIG. 5.

In a possible design, the MOC 12 is connected to the key member 13 through the control switch 14. The MOC 12 is specifically configured to control the key member 13 to be in a power-down state through the control switch 14, when the MOC is in a power-up state.

In a possible design, the MOC 12 has a GPIO interface, and the MOC 12 can control a state of the control switch 14 between the MOC 12 and the key member 13 through the GPIO interface.

The embodiment of the present disclosure provides a fingerprint identification system, where the fingerprint identification system includes the fingerprint module described in the above embodiments, and reference can be made to the embodiments of the fingerprint module for effects of the fingerprint identification system, which will not be repeated here.

On the basis of the previous embodiment, further, when the main control unit does not receive information, for example, the authentication result sent by the MOC 12, within a preset period of time, the main control unit sends a second control instruction to the MOC 12 and/or the fingerprint sensor 11, so that the MOC 12 and/or the fingerprint sensor 11 enters the power-down state according to the second control instruction. The above preset period of time can be set according to an actual condition, for example, the preset period is set to 10 minutes, 15 minutes, etc., which is not limited in the present disclosure.

In a possible design, when the MOC 12 enters the power-down state, the MOC 12 is further configured to control the key member 13 to be in the power-up state.

In a possible design, the MOC 12 is connected to the key member 13 through the control switch 14, and when the MOC 12 is in the power-down state, the MOC 12 is configured to trigger the control switch 14 to output a low-level signal, where the low-level signal is used to trigger the key member to be in the power-up state.

Or, in another possible design, when the MOC 12 is in the power-down state, the MOC 12 is configured to trigger the control switch 14 to output a high-level signal; where the high-level signal is used to trigger the key member to be in the power-up state.

In the above solution, the MOC 12 is configured to control the key member 13 to be in the power-up state. Actually, the main control unit may also directly control the key member 13 to be in the power-up state.

In a possible design, the main control unit is connected to the key member through the control switch, and when the main control unit is in the sleep state, the main control unit triggers the control switch to output a low-level signal, where the low-level signal is used to trigger the key member to be in the power-up state.

Or, in another possible design, when the main control unit is in the sleep state, the main control unit triggers the control switch to output a high-level signal, where the high-level signal is used to trigger the key member to be in the power-up state.

The embodiment of the present disclosure provides a fingerprint identification system, where the fingerprint identification system includes the fingerprint module described in the above embodiments, and reference can be made to the embodiments of the fingerprint module for effects of the fingerprint identification system, which will not be repeated here.

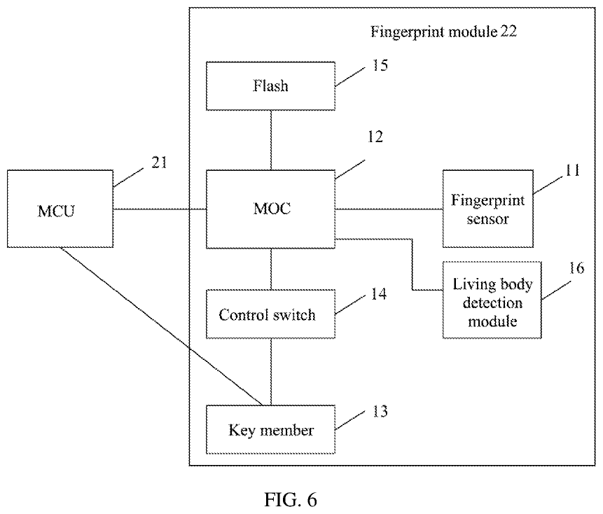

On the basis of any of the previous embodiments, further, the fingerprint module further includes: a flash and/or a living body detection module. The following is a detailed description of the fingerprint module including a flash and/or a living body detection module. FIG. 6 is a schematic diagram of a fingerprint identification system according to still another embodiment of the present disclosure. As shown in FIG. 6, the fingerprint module in the system further includes: a flash 15 and a living body detection module 16. The flash 15 includes at least one fingerprint template, and correspondingly, the MOC 12 is specifically configured to match the fingerprint information with the at least one fingerprint template, and output an authentication result to the main control unit. The authentication result is a result of successful authentication or a result of failed authentication.

In a possible design, the living body detection module 16 is configured to detect at least one living body index of the verification target, and output the at least one living body index to the MOC 12. The MOC 12 is further configured to determine whether the verification target is a living body according to the at least one living body index. The living body index includes at least one of: blood oxygen or heart rate. For example: when determining that a heart rate of the verification target is within a preset range, the MOC 12 determines the verification target is a living body.

When the fingerprint information is successfully authenticated and the verification target is the living body, the MOC 12 may output an authentication result of successful authentication to the main control unit 21. Conversely, when the fingerprint information fails to be authenticated or the verification target is not the living body, the MOC 12 may output an authentication result of failed authentication to the main control unit.

In a possible design, after the main control unit 21 is woken up, in this case, if at least one of the MOC 12, the fingerprint sensor 11, the flash 15 and the living body detection module 16 is in the power-off state, the main control unit 21 will send a first control instruction to at least one of the MOC 12, the fingerprint sensor 11, the flash 15 and the living body detection module 16, so that at least one of the MOC 12, the fingerprint sensor 11, the flash 15 and the living body detection module 16 will enter the power-up state according to the first control instruction.

When the main control unit 21 does not receive information, for example, the authentication result sent by the MOC 12, within a preset period of time, and at least one of the MOC 12, the fingerprint sensor 11, the flash 15 and the living body detection module 16 is in the power-up state, the main control unit 21 may send a second control instruction to at least one of the MOC 12, the fingerprint sensor 11, the flash 15 and the living body detection module 16, so that at least one of the MOC 12, the fingerprint sensor 11, the flash 15 and the living body detection module 16 enters the power-down state according to the second control instruction.

The embodiment of the present disclosure provides a fingerprint identification system, where the fingerprint identification system includes the fingerprint module described in the above embodiments, and reference can be made to the embodiments of the fingerprint module for effects of the fingerprint identification system, which will not be repeated here.

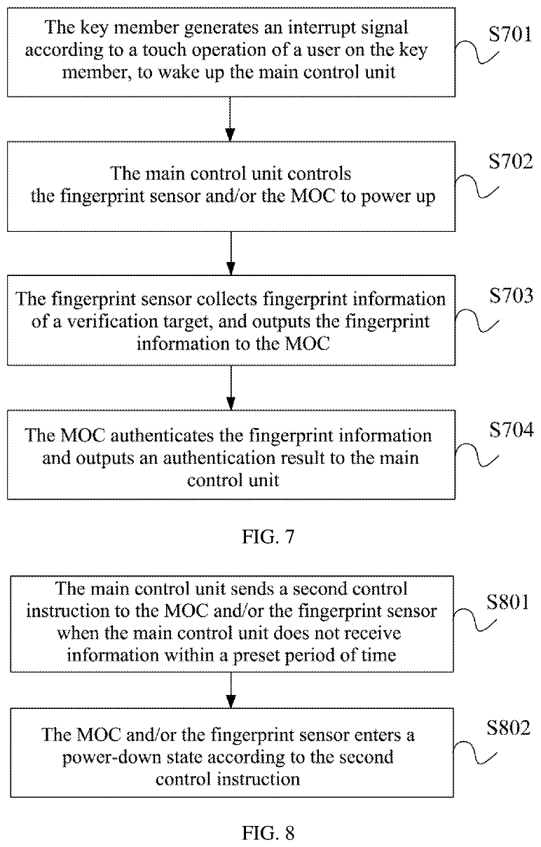

FIG. 7 is a flowchart of a control method based on a fingerprint identification system according to an embodiment of the present disclosure. The fingerprint identification system includes: a main control unit and a fingerprint module. The fingerprint module includes: a fingerprint sensor, a match on chip (MOC) and a key member. The key member is connected to the fingerprint sensor. As shown in FIG. 7, the method includes the following process:

Step S701: The key member generates an interrupt signal according to a touch operation of a user on the key member, to wake up the main control unit.

Step S702: The main control unit controls the fingerprint sensor and/or the MOC to power up.

Step S703: The fingerprint sensor collects fingerprint information of a verification target, and outputs the fingerprint information to the MOC.

Step S704: The MOC authenticates the fingerprint information and outputs an authentication result to the main control unit.

In a possible design, step S702 includes: the main control unit sends a first control instruction to the MOC and/or the fingerprint sensor, so that the MOC and/or the fingerprint sensor enters a power-up state according to the first control instruction.

In a possible design, the method further includes: the MOC controls the key member to be in a power-down state, when the MOC is in a power-up state.

In a possible design, the MOC is connected to the key member through a control switch; and the MOC controlling, the key member to be in a power-down state includes: the MOC controls the key member to be in the power-down state through the control switch, when the MOC is in the power-up state.

The method may be executed by the above fingerprint identification system, and for contents and effects thereof, reference may be made to the embodiments of the above fingerprint identification system, which will not be repeated here.

FIG. 8 is a flowchart of a control method based on a fingerprint identification system according to another embodiment of the present disclosure. The fingerprint identification system includes: a main control unit and a fingerprint module. The fingerprint module includes: a fingerprint sensor, an MOC and a key member. The key member is connected to the fingerprint sensor. As shown in FIG. 8, the method includes the following process:

Step S801: The main control unit sends a second control instruction to the MOC and/or the fingerprint sensor when the main control unit does not receive information within a preset period of time.

Step S802: The MOC and/or the fingerprint sensor enters a power-down state according to the second control instruction.

In a possible design, when the MOC is in the power-down state, the method further includes: the MOC controls the key member to be in a power-up state.

In a possible design, the MOC is connected to the key member through a control switch, and the MOC controlling the key member to be in a power-up state includes: the MOC triggers the control switch to output a low-level signal when the MOC is in the power-down state, where the low-level signal is used to trigger the key member to be in the power-up state.

In a possible design, the fingerprint module further includes: a flash; the flash includes at least one fingerprint template; the MOC authenticating the fingerprint information and outputting the authentication result to the main control unit includes: the MOC matches the fingerprint information with the at least one fingerprint template, and outputs the authentication result.

In a possible design, the authentication result is a result of successful authentication or a result of failed authentication.

In a possible design, the fingerprint module further includes a living body detection module; and correspondingly, the method further includes: the living body detection module detects at least one living body index of the verification target, and outputs the at least one living body index to the MOC; and the MOC determines whether the verification target is a living body according to the at least one living body index.

In a possible design, there further includes: the MOC outputs an authentication result of successful authentication when the fingerprint information is successfully authenticated and the verification target is a living body.

In a possible design, the living body index includes at least one of: blood oxygen or heart rate.

The method may be performed by the above fingerprint identification system, and for contents and effects thereof, reference may be made to the embodiments of the above fingerprint identification system, which will not be repeated here.

The present disclosure further provides a smart lock, including: the above fingerprint identification system. For functions and effects of the system, reference may be made to the above embodiments, which will not be repeated in the present disclosure.

It should be noted that an unlocking method of the smart lock may include a fingerprint password recognition method. Optionally, the smart lock may further include at least one unlocking method as follows: an unlocking method based on NFC, a digital password unlocking method, and the like.

Persons of ordinary skill in the art could understand that all or part of the steps in the above method embodiments may be implemented by a program instructing relevant hardware. The foregoing program may be stored in a computer readable storage medium. When the program is executed, the steps including the above method embodiments are executed. The foregoing storage medium may include a ROM, a RAM, a magnetic disk, or an optical disk and other media that can store program codes.

Finally, it should be noted that the above embodiments are merely used to describe the technical solutions of the present disclosure other than limiting the present disclosure. Although the present disclosure is described in detail with reference to the foregoing embodiments, persons of ordinary skill in the art should understand that they may still make modifications to the technical solutions described in the foregoing embodiments or make equivalent substitutions to all or some technical features thereof. However, these modifications and substitutions do not cause the corresponding technical solutions departing from the technical solutions of the embodiments of the present disclosure.

What is claimed is:

1. A fingerprint module, connected to a main control unit, the fingerprint module comprising: a fingerprint sensor, a match on chip (MOC) and a key member, and the key member is connected to the fingerprint sensor;
the fingerprint sensor is configured to collect fingerprint information of a verification target, and output the fingerprint information to the MOC;
the MOC is configured to authenticate the fingerprint information, and output an authentication result to the main control unit; and
the key member is configured to generate an interrupt signal according to a touch operation of a user on the key member, to wake up the main control unit.

2. The fingerprint module according to claim 1, wherein:
at least one of the MOC or the fingerprint sensor is further configured to receive a first control instruction sent by the main control unit, and enter a power-up state according to the first control instruction.

3. The fingerprint module according to claim 2, wherein:
the MOC is further configured to control the key member to be in a power-down state.

4. The fingerprint module according to claim 3, wherein the MOC is connected to the key member through a control switch; and
the MOC is configured to control the key member to be in the power-down state through the control switch when the MOC is in the power-up state.

5. The fingerprint module according to claim 1, wherein:
at least one of the MOC or the fingerprint sensor is further configured to receive a second control instruction sent by the main control unit, and enter a power-down state according to the second control instruction.

6. The fingerprint module according to claim 5, wherein:
the MOC is further configured to control the key member to be in a power-up state.

7. The fingerprint module according to claim 6, wherein the MOC is connected to the key member through a control switch; and
the MOC is configured to trigger the control switch to output a low-level signal when the MOC is in the power-down state; and
the low-level signal is used to trigger the key member to be in the power-up state.

8. The fingerprint module according to claim 1, further comprising: a flash,
the flash comprising at least one fingerprint template; and
the MOC being configured to match the fingerprint information with the at least one fingerprint template, and output an authentication result.

9. The fingerprint module according to claim 8, wherein:
the authentication result is a result of successful authentication or a result of failed authentication.

10. The fingerprint module according to claim 1, further comprising a living body detection module,
the living body detection module is configured to detect at least one living body index of the verification target, and output the at least one living body index to the MOC; and
the MOC is further configured to determine whether the verification target is a living body according to the at least one living body index.

11. The fingerprint module according to claim 10, wherein the MOC is configured to output an authentication result of successful authentication when the fingerprint information is successfully authenticated and the verification target is the living body.

12. The fingerprint module according to claim 10, wherein the living body index comprises at least one of: blood oxygen or heart rate.

13. A fingerprint identification system, comprising: a main control unit and the fingerprint module according to claim 1.

14. The fingerprint identification system according to claim 13, wherein:
the main control unit is configured to: send a second control instruction to at least one of the MOC or the fingerprint sensor when the main control unit does not receive information within a preset period of time, and the second control instruction is configured to control at least one of the MOC or the fingerprint sensor to enter a power-down state.

15. A control method based on a fingerprint identification system, the fingerprint identification system including a main control unit and a fingerprint module, and the fingerprint module includes a fingerprint sensor, a match on chip (MOC) and a key member, and the key member is connected to the fingerprint sensor; and correspondingly, the control method comprising:
generating, by the key member, an interrupt signal according to a touch operation of a user on the key member, to wake up the main control unit;
controlling, by the main control unit, at least one of the fingerprint sensor or the MOC to power up;
collecting, by the fingerprint sensor, fingerprint information of a verification target, and outputting the fingerprint information to the MOC; and
authenticating, by the MOC, the fingerprint information and outputting an authentication result to the main control unit.

16. The control method according to claim 15, wherein the controlling, by the main control unit, at least one of the fingerprint sensor or the MOC to power up, comprises:
sending, by the main control unit, a first control instruction to at least one of the MOC or the fingerprint sensor, so that at least one of the MOC or the fingerprint sensor enters a power-up state according to the first control instruction.

17. The control method according to claim 15, further comprising:
sending, by the main control unit, a second control instruction to at least one of the MOC or the fingerprint sensor, when the main control unit does not receive information within a preset period of time; and
entering, by at least one of the MOC or the fingerprint sensor, a power-down state according to the second control instruction.

18. The control method according to claim 15, wherein the fingerprint module further comprises: a flash, the flash comprising at least one fingerprint template;
the authenticating, by the MOC, of the fingerprint information and outputting an authentication result to the main control unit, comprises:
matching, by the MOC, of the fingerprint information with the at least one fingerprint template, and outputting the authentication result; and
the authentication result is a result of successful authentication or a result of failed authentication.

19. The control method according to claim 15, wherein the fingerprint module further comprises a living body detection module; and the control method further comprises:
detecting, by the living body detection module, at least one living body index of the verification target, and outputting the at least one living body index to the MOC;
determining, by the MOC, whether the verification target is a living body according to the at least one living body index; and
the control method further comprises:
outputting, by the MOC, an authentication result of successful authentication, when the fingerprint information is successfully authenticated and the verification target is a living body.

20. A smart lock, comprising: the fingerprint identification system according to claim 13.
